(19) United States
(12) Patent Application Publication
(10) Pub. No.: US 2025/0382358 A1
(43) Pub. Date: Dec. 18, 2025

(54) METHODS AND COMPOSITIONS RELATING TO MOUSE MONOCLONAL ANTI-G4S LINKER ANTIBODIES

(71) Applicant: BioLegend, Inc., San Diego, CA (US)

(72) Inventors: Anil Kumar JAISWAL, San Diego, CA (US); Nicolas Mathieu Schrantz, San Diego, CA (US); Fen-Ling Chen, Hsinchu (TW); Weihao Chen, Hsinchu (TW); Chien-Chung Chen, Hsinchu (TW)

(21) Appl. No.: 19/234,949

(22) Filed: Jun. 11, 2025

Related U.S. Application Data
(60) Provisional application No. 63/661,204, filed on Jun. 18, 2024.

(51) Int. Cl. C07K 16/18; G01N 33/569; G01N 33/58

(52) U.S. Cl. CPC C07K 16/18; G01N 33/56972; G01N 33/58

(57) ABSTRACT

Mouse monoclonal antibodies are provided according to aspects of the present disclosure which specifically bind to G4S(n). Mouse monoclonal antibodies which specifically bind to G4S(n) are disclosed which include particular heavy chain variable region complementarity determining regions, light chain variable region complementarity determining regions, and variants thereof. Methods for detecting G4S(n) in a sample according to aspects of the present disclosure include contacting a sample containing or suspected of containing G4S(n) with a mouse monoclonal anti-G4S(n) antibody, under binding conditions; and detecting binding of the anti-G4S(n) antibody thereof with G4S(n). According to aspects of the present disclosure, the sample comprises CAR-T cells, wherein the CAR-T cells comprise an expression construct encoding a G4S(n) adjacent to a chimeric antigen receptor. Immunoassay kits including one or more mouse monoclonal antibodies which specifically bind to G4S(n) of the present disclosure are described herein.

### REFERENCE TO RELATED APPLICATION

This application claims priority from U.S. Provisional Patent Application Ser. No. 63/661,204, filed Jun. 18, 2024, the entire content of which is incorporated herein by reference.

### SEQUENCE LISTING

The instant application contains a Sequence Listing which has been submitted electronically in XML format and is hereby incorporated by reference in its entirety. Said XML format Sequence Listing, created on Jun. 10, 2025, is named REVB0003US_68463-0024.xml and is 29,861 bytes in size.

### FIELD OF THE INVENTION

Generally described are mouse monoclonal antibodies and methods of their use relating to detection of G4S linkers. Specifically described are mouse monoclonal antibodies which specifically bind to G4S₍ₙ₎ linkers, and methods of their use.

### BACKGROUND OF THE INVENTION

CAR-T cell therapy requires genetic modification of a patient's T cells. Specifically, an expression construct encoding a chimeric antigen receptor (CAR) is introduced into T cells isolated from the patient so that the modified T cells will express the CAR such that the T cells will bind to, and kill, cancer cells in the patient.

While CAR-T therapy has shown significant value as a therapeutic approach, currently there are no or limited resources available to detect the CAR-T cells in research or clinical settings.

There is a continuing need for compositions and methods for detecting CAR-T cells.

### BRIEF SUMMARY

Mouse monoclonal antibodies are provided according to aspects of the present disclosure which specifically bind to G4S(n).

Mouse monoclonal antibodies according to aspects of the present disclosure include heavy chain variable region complementarity determining regions (VHCDR): VHCDR 1 of SEQ ID NO: 1, VHCDR2 of SEQ ID NO:2, and VHCDR3 of SEQ ID NO:3, or a variant of any one, two or three of SEQ ID NO: 1, SEQ ID NO:2, or SEQ ID NO:3.

Mouse monoclonal antibodies according to aspects of the present disclosure include light chain variable region complementarity determining regions (VLCDR): VLCDR1 of SEQ ID NO: 4, VLCDR2 of SEQ ID NO:5, and VLCDR3 of SEQ ID NO:6, or a variant of any one, two, or three SEQ ID NO: 4, SEQ ID NO:5, or SEQ ID NO:6.

Mouse monoclonal antibodies according to aspects of the present disclosure include heavy chain variable region complementarity determining regions (VHCDR): VHCDR1 of SEQ ID NO: 1, VHCDR2 of SEQ ID NO:2, and VHCDR3 of SEQ ID NO:3, or a variant of any one, two or three of SEQ ID NO: 1, SEQ ID NO:2, or SEQ ID NO:3; and include light chain variable region complementarity determining regions (VLCDR): VLCDR1 of SEQ ID NO: 4, VLCDR2 of SEQ ID NO:5, and VLCDR3 of SEQ ID NO:6, or a variant of any one, two, or three SEQ ID NO: 4, SEQ ID NO:5, or SEQ ID NO:6.

Mouse monoclonal antibodies according to aspects of the present disclosure include a heavy chain variable region (VH) of SEQ ID NO: 7, or a variant thereof.

Mouse monoclonal antibodies according to aspects of the present disclosure include a light chain variable region (VL) of SEQ ID NO: 8, or a variant thereof.

Mouse monoclonal antibodies according to aspects of the present disclosure include a heavy chain variable region (VH) of SEQ ID NO: 7, or a variant thereof; and a light chain variable region (VL) of SEQ ID NO: 8, or a variant thereof.

Mouse monoclonal antibodies according to aspects of the present disclosure include a heavy chain variable region (VH) encoded by SEQ ID NO:17, or a variant thereof.

Mouse monoclonal antibodies according to aspects of the present disclosure include a light chain variable region (VL) encoded by SEQ ID NO: 18, or a variant thereof.

Mouse monoclonal antibodies according to aspects of the present disclosure include a heavy chain variable region (VH) encoded by SEQ ID NO:17, or a variant thereof; and a light chain variable region (VL) encoded by SEQ ID NO:18, or a variant thereof.

Mouse monoclonal antibodies according to aspects of the present disclosure include heavy chain variable region complementarity determining regions (VHCDR): VHCDR1 of SEQ ID NO: 9, VHCDR2 of SEQ ID NO: 10, and VHCDR3 of SEQ ID NO: 11, or a variant of any one, two or three of SEQ ID NO: 9, SEQ ID NO: 10, or SEQ ID NO: 11.

Mouse monoclonal antibodies according to aspects of the present disclosure include light chain variable region complementarity determining regions (VLCDR): VLCDR1 of SEQ ID NO: 12, VLCDR2 of SEQ ID NO:13, and VLCDR3 of SEQ ID NO: 14, or a variant of any one, two, or three SEQ ID NO: 12, SEQ ID NO: 13, or SEQ ID NO14.

Mouse monoclonal antibodies according to aspects of the present disclosure include heavy chain variable region complementarity determining regions (VHCDR): VHCDR1 of SEQ ID NO: 9, VHCDR2 of SEQ ID NO:10, and VHCDR3 of SEQ ID NO: 11, or a variant of any one, two or three of SEQ ID NO: 9, SEQ ID NO: 10, or SEQ ID NO: 11; and light chain variable region complementarity determining regions (VLCDR): VLCDR1 of SEQ ID NO: 12, VLCDR2 of SEQ ID NO:13, and VLCDR3 of SEQ ID NO: 14, or a variant of any one, two, or three SEQ ID NO: 12, SEQ ID NO: 13, or SEQ ID NO14.

Mouse monoclonal antibodies according to aspects of the present disclosure include a heavy chain variable region (VH) of SEQ ID NO: 15, or a variant thereof.

Mouse monoclonal antibodies according to aspects of the present disclosure include a light chain variable region (VL) of SEQ ID NO: 16, or a variant thereof.

Mouse monoclonal antibodies according to aspects of the present disclosure include a heavy chain variable region (VH) of SEQ ID NO: 15, or a variant thereof; and a light chain variable region (VL) of SEQ ID NO: 16, or a variant thereof.

Mouse monoclonal antibodies according to aspects of the present disclosure include a heavy chain variable region (VH) encoded by SEQ ID NO: 19, SEQ ID NO:21, or a variant or either thereof.

Mouse monoclonal antibodies according to aspects of the present disclosure include a light chain variable region (VL) encoded by SEQ ID NO:20, SEQ ID NO:22, or a variant of either thereof.

Mouse monoclonal antibodies according to aspects of the present disclosure include a heavy chain variable region (VH) encoded by SEQ ID NO:19, SEQ ID NO:21, or a variant or either thereof; and a light chain variable region (VL) encoded by SEQ ID NO: 20, SEQ ID NO:22, or a variant of either thereof.

Mouse monoclonal antibodies according to aspects of the present disclosure include a variant of heavy chain variable region 1 encoded by a nucleotide sequence which hybridizes to the complement of SEQ ID NO: 17, under stringent hybridization conditions.

Mouse monoclonal antibodies according to aspects of the present disclosure include a variant of light chain variable region 1 encoded by a nucleotide sequence which hybridizes to the complement of SEQ ID NO: 18, under stringent hybridization conditions.

Mouse monoclonal antibodies according to aspects of the present disclosure include a variant of heavy chain variable region 1 encoded by a nucleotide sequence which hybridizes to the complement of SEQ ID NO: 19, or SEQ ID NO: 21, under stringent hybridization conditions.

Mouse monoclonal antibodies according to aspects of the present disclosure include a variant of light chain variable region 1 encoded by a nucleotide sequence which hybridizes to the complement of SEQ ID NO: 20, or SEQ ID NO: 22, under stringent hybridization conditions.

Mouse monoclonal antibodies according to aspects of the present disclosure include a detectable label.

Expression cassettes according to aspects of the present disclosure include a nucleic acid encoding any of: heavy chain variable region complementarity determining regions (VHCDR): VHCDR1 of SEQ ID NO: 9, VHCDR2 of SEQ ID NO:10, and VHCDR3 of SEQ ID NO: 11, or a variant of any one, two or three of SEQ ID NO: 9, SEQ ID NO: 10, or SEQ ID NO:11; light chain variable region complementarity determining regions (VLCDR): VLCDR1 of SEQ ID NO: 12, VLCDR2 of SEQ ID NO:13, and VLCDR3 of SEQ ID NO: 14, or a variant of any one, two, or three SEQ ID NO: 12, SEQ ID NO: 13, or SEQ ID NO14; VHCDR1 of SEQ ID NO: 9, VHCDR2 of SEQ ID NO:10, and VHCDR3 of SEQ ID NO: 11, or a variant of any one, two or three of SEQ ID NO: 9, SEQ ID NO:10, or SEQ ID NO:11; and light chain variable region complementarity determining regions (VLCDR): VLCDR1 of SEQ ID NO: 12, VLCDR2 of SEQ ID NO: 13, and VLCDR3 of SEQ ID NO:14, or a variant of any one, two, or three SEQ ID NO: 12, SEQ ID NO:13, or SEQ ID NO14.

Expression cassettes according to aspects of the present disclosure include a nucleic acid encoding a heavy chain variable region (VH) of SEQ ID NO: 15, or a variant thereof.

Expression cassettes according to aspects of the present disclosure include a nucleic acid encoding a light chain variable region (VL) of SEQ ID NO: 16, or a variant thereof.

Expression cassettes according to aspects of the present disclosure include a nucleic acid encoding a heavy chain variable region (VH) of SEQ ID NO: 15, or a variant thereof; and a nucleic acid encoding a light chain variable region (VL) of SEQ ID NO: 16, or a variant thereof.

Expression cassettes according to aspects of the present disclosure include a nucleic acid encoding a heavy chain variable region (VH) encoded by SEQ ID NO:19, SEQ ID NO:21, or a variant or either thereof.

Expression cassettes according to aspects of the present disclosure include a nucleic acid encoding a light chain variable region (VL) encoded by SEQ ID NO:20, SEQ ID NO: 22, or a variant of either thereof.

Expression cassettes according to aspects of the present disclosure include a nucleic acid encoding a heavy chain variable region (VH) encoded by SEQ ID NO:19, SEQ ID NO:21, or a variant or either thereof; and a light chain variable region (VL) encoded by SEQ ID NO:20, SEQ ID NO:22, or a variant of either thereof.

Expression vectors according to aspects of the present disclosure include an expression cassette which includes a nucleic acid encoding any of: heavy chain variable region complementarity determining regions (VHCDR): VHCDR1 of SEQ ID NO: 9, VHCDR2 of SEQ ID NO: 10, and VHCDR3 of SEQ ID NO:11, or a variant of any one, two or three of SEQ ID NO: 9, SEQ ID NO:10, or SEQ ID NO:11; light chain variable region complementarity determining regions (VLCDR): VLCDR1 of SEQ ID NO: 12, VLCDR2 of SEQ ID NO: 13, and VLCDR3 of SEQ ID NO:14, or a variant of any one, two, or three SEQ ID NO: 12, SEQ ID NO:13, or SEQ ID NO14; VHCDR1 of SEQ ID NO: 9, VHCDR2 of SEQ ID NO: 10, and VHCDR3 of SEQ ID NO: 11, or a variant of any one, two or three of SEQ ID NO: 9, SEQ ID NO: 10, or SEQ ID NO:11; and light chain variable region complementarity determining regions (VLCDR): VLCDR1 of SEQ ID NO: 12, VLCDR2 of SEQ ID NO: 13, and VLCDR3 of SEQ ID NO: 14, or a variant of any one, two, or three SEQ ID NO: 12, SEQ ID NO: 13, or SEQ ID NO14.

Expression vectors according to aspects of the present disclosure include an expression cassette which includes a nucleic acid encoding a heavy chain variable region (VH) of SEQ ID NO: 15, or a variant thereof.

Expression vectors according to aspects of the present disclosure include an expression cassette which includes a nucleic acid encoding a light chain variable region (VL) of SEQ ID NO: 16, or a variant thereof.

Expression vectors according to aspects of the present disclosure include an expression cassette which includes a nucleic acid encoding a heavy chain variable region (VH) of SEQ ID NO: 15, or a variant thereof; and a nucleic acid encoding a light chain variable region (VL) of SEQ ID NO: 16, or a variant thereof.

Expression vectors according to aspects of the present disclosure include an expression cassette which includes a nucleic acid encoding a heavy chain variable region (VH) encoded by SEQ ID NO:19, SEQ ID NO:21, or a variant or either thereof.

Expression vectors according to aspects of the present disclosure include an expression cassette which includes a nucleic acid encoding a light chain variable region (VL) encoded by SEQ ID NO:20, SEQ ID NO:22, or a variant of either thereof.

Expression vectors according to aspects of the present disclosure include an expression cassette which includes a nucleic acid encoding a heavy chain variable region (VH) encoded by SEQ ID NO: 19, SEQ ID NO:21, or a variant or either thereof; and a light chain variable region (VL) encoded by SEQ ID NO:20, SEQ ID NO:22, or a variant of either thereof.

Host cells including an expression vector according to aspects of the present disclosure include an expression cassette which includes a nucleic acid encoding a heavy chain variable region (VH) of SEQ ID NO: 15, or a variant thereof.

Host cells including an expression vector according to aspects of the present disclosure include an expression cassette which includes a nucleic acid encoding a light chain variable region (VL) of SEQ ID NO: 16, or a variant thereof.

Host cells including an expression vector according to aspects of the present disclosure include an expression cassette which includes a nucleic acid encoding a heavy chain variable region (VH) of SEQ ID NO: 15, or a variant thereof; and a nucleic acid encoding a light chain variable region (VL) of SEQ ID NO: 16, or a variant thereof.

Host cells including an expression vector according to aspects of the present disclosure include an expression cassette which includes a nucleic acid encoding a heavy chain variable region (VH) encoded by SEQ ID NO:19, SEQ ID NO:21, or a variant or either thereof.

Host cells including an expression vector according to aspects of the present disclosure include an expression cassette which includes a nucleic acid encoding a light chain variable region (VL) encoded by SEQ ID NO:20, SEQ ID NO:22, or a variant of either thereof.

Host cells including an expression vector according to aspects of the present disclosure include an expression cassette which includes a nucleic acid encoding a heavy chain variable region (VH) encoded by SEQ ID NO:19, SEQ ID NO:21, or a variant or either thereof; and a light chain variable region (VL) encoded by SEQ ID NO:20, SEQ ID NO: 22, or a variant of either thereof.

Methods for detecting G4S(n) in a sample according to aspects of the present disclosure include: contacting a sample containing or suspected of containing G4S(n) with a mouse monoclonal anti-G4S(n) antibody, under binding conditions; and detecting binding of the anti-G4S(n) antibody thereof with G4S(n). According to aspects of the present disclosure, the sample comprises CAR-T cells, wherein the CAR-T cells comprise an expression construct encoding a G4S(n) adjacent to a chimeric antigen receptor. According to aspects present disclosure, the method includes immunochromatography; antigen capture; flow cytometry; immunoblot; immunofluorescence; immunoprecipitation; immunodiffusion; competitive immunoassay, immunocytochemistry; radioimmunoassay; and combinations of any of these. According to aspects of the present disclosure, the sample contains human CAR-T cells.

ELISA methods for detecting G4S(n) in a sample according to aspects of the present disclosure include: contacting a sample containing or suspected of containing G4S(n) with a mouse monoclonal anti-G4S(n) antibody, under binding conditions; and detecting binding of the anti-G4S(n) antibody thereof with G4S(n). According to aspects of the present disclosure, the sample comprises CAR-T cells, wherein the CAR-T cells comprise an expression construct encoding a G4S(n) adjacent to a chimeric antigen receptor. According to aspects of the present disclosure, the sample contains human CAR-T cells.

Immunoassay kits are provided according to aspects of the present disclosure which include one or more antibodies, one or more expression cassettes, one or more expression vectors, and/or one or more host cells described herein.

### BRIEF DESCRIPTION OF THE DRAWINGS

FIGS. 1A and 1B show graphs representing results of flow cytometry immunoassays assessing binding of mouse monoclonal anti-G4S₍ₙ₎ linker antibody C in amounts of 0.5 ug, 1.0 ug, and 2.0 ug compared to the same amounts of an isotype control (RTK2758) on G4S₍ₙ₎ linker-transfected CHO cells;

FIG. 2 shows graphs representing results of flow cytometry assays assessing co-staining of mouse monoclonal anti-G4S₍ₙ₎ linker antibody C or A with anti-HA.11 antibody; or isotype control antibody RTK2758 or MPC-11, with anti-HA.11 antibody; on G4S₍ₙ₎ linker-transfected CHO cells;

FIG. 3 shows graphs representing results of flow cytometry assays assessing co-staining of mouse monoclonal anti-G4S₍ₙ₎ linker antibody C or A with anti-HA.11 antibody; or isotype control antibody RTK2758 or MPC-11, with anti-HA.11 antibody; on G4S₍ₙ₎ linker-transfected CHO cells;

FIG. 4 shows graphs representing results of flow cytometry assays assessing co-staining of mouse monoclonal anti-G4S₍ₙ₎ linker antibody C with anti-HA.11 antibody; or isotype control antibody RTK2758 or MPC-21, with anti-HA.11 antibody; on G4S₍ₙ₎ linker-transfected CHO cells;

FIG. 5 shows results of flow cytometry assays assessing immunostaining of K562 cells transfected with an expression construct encoding a G4S₍ₙ₎ linker and green fluorescent protein (GFP);

FIG. 6 shows results of flow cytometry assays assessing immunostaining of K562 cells transfected with an expression construct encoding a G4S₍ₙ₎ linker and green fluorescent protein (GFP); and

FIG. 7 shows histograms representing single parameter analysis of results of flow cytometry assays assessing immunostaining of mouse monoclonal anti-G4S₍ₙ₎ linker antibody C or A; or isotype control antibody RTK2758 or MPC-11; on G4S₍ₙ₎ linker-transfected K562 cells.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Herdewijn, P. (Ed.), Oligonucleotide Synthesis: Methods and Applications, Methods in Molecular Biology, Humana Press, 2004; Kursad Turksen (Ed.), Embryonic stem cells: methods and protocols in Methods Mol Biol. 2002; 185, Humana Press; Current Protocols in Stem Cell Biology, ISBN: 9780470151808; Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

The singular terms “a,” “an,” and “the” are not intended to be limiting and include plural referents unless explicitly stated otherwise or the context clearly indicates otherwise.

Compositions and methods for detecting CAR-T cells are provided according to aspects of the present disclosure which include detection of a G4S₍ₙ₎ linker adjacent to a chimeric antigen receptor in a protein construct in vivo (such as in a cell), or in vitro (such as in isolated recombinant protein). According to aspects of the present disclosure, antibodies, kits, and methods for detecting CAR-T cells are provided which allow for, or include, detection of a G4S₍ₙ₎ linker adjacent to a chimeric antigen receptor in a recombinant protein construct in a T cell obtained from a subject.

According to aspects of the present disclosure, antibodies, kits, and methods for detecting CAR-T cells are provided which allow for, or include, detection of a G4S₍ₙ₎ linker adjacent to a chimeric antigen receptor in a recombinant protein construct in a T cell obtained from a human subject.

According to aspects of the present disclosure, detection of a G4S₍ₙ₎ linker adjacent to a chimeric antigen receptor in a recombinant protein construct is accomplished by immunoassay using an antibody disclosed herein which specifically binds G4S₍ₙ₎. The detection of a G4S₍ₙ₎ linker adjacent to a chimeric antigen receptor in a recombinant protein construct allows a user to detect CAR-T cells.

### Antibodies

Antibodies are provided according to aspects of the present disclosure which specifically bind to a G4S₍ₙ₎ linker with high affinity. Mouse monoclonal antibodies are provided according to aspects of the present disclosure which specifically bind to a G4S₍ₙ₎ linker with high affinity. The terms “anti-G4S₍ₙ₎ antibody” and “anti-G4S₍ₙ₎ mouse monoclonal antibody” as used herein to refer to antibodies of the present disclosure, which specifically bind to G4S₍ₙ₎ with high affinity.

The terms “G4G4S₍ₙ₎” and “G4S₍ₙ₎ linker” as used herein refer to a poly-Glycine-Serine peptide linker characterized by a pentapeptide amino acid sequence GGGGS (“G4S”, “Gly4Ser”, “GlyGlyGlyGlySer”) (SEQ ID NO:29), repeated “n” times where n is 1, 2, 3, or 4, i.e. GGGGS (SEQ ID NO:29), GGGGSGGGGS (SEQ ID NO:30), GGGGSGGGGSGGGGS (SEQ ID NO:31), or GGGGSGGGGSGGGGSGGGGS (SEQ ID NO: 32). The sequence G4S₍ₙ₎ may itself constitute the entire linker or it may be part of a larger linker.

As used herein, the terms “antibody” and “antibodies” relate to mouse monoclonal antibodies and antigen-binding fragments of mouse monoclonal antibodies. In particular aspects, antibodies include mouse immunoglobulin molecules and immunologically active fragments of immunoglobulin molecules, i.e., molecules that contain an antigen binding site. Immunoglobulin molecules are of any type (e.g., IgG, IgE, IgM, IgD, and IgA), class or subclass (e.g., IgG1, IgG2 including IgG2a and IgG2b, IgG3, IgG4, IgA1 and IgA2).

The term “monoclonal antibody” as used herein refers to an antibody obtained from a substantially homogeneous population of antibodies. The phrase “substantially homogeneous population of antibodies” indicates that the antibodies of the homogeneous population are identical except that variants may occur, such as variants due to mutations that occur during production, or post-translational variations. A variant retains at least a substantial proportion, at least about 50%, 60%, 70%, 80%, 90%, 95%, 98%, 99% or greater %, of the affinity/avidity and/or the specificity/selectivity of a reference antibody Antibody A, Antibody B, or Antibody C disclosed herein. According to aspects of the present disclosure, a variant retains at least a substantial proportion, at least about 95%, 96%, 97%, 98%, 99%, or greater %, of the affinity/avidity and/or the specificity/selectivity of a reference antibody Antibody A, Antibody B, or Antibody C disclosed herein. The term monoclonal antibody is not limited to monoclonal antibodies produced using any particular method. Monoclonal antibodies of the present disclosure may be produced using hybridoma methodology, recombinant methodology, phage display methodology, synthetic methodology, other monoclonal methodologies known in the art, or combinations of any two or more thereof.

As used herein, the term “antigen-binding fragment” defines a fragment of a mouse monoclonal antibody that immunospecifically binds to a target antigen. An antigen-binding fragment may be generated by any technique known to one of skill in the art. For example, Fab and F(ab′)₂ antigen-binding fragments may be produced by proteolytic cleavage of immunoglobulin molecules, using enzymes such as papain (to produce Fab antigen-binding fragments) or pepsin (to produce F(ab′)₂ antigen-binding fragments). Antigen-binding fragments are also produced by recombinant DNA technologies. Antigen-binding fragments encompassed by the present compositions, kits, and methods possess the ability to specifically bind G4S₍ₙ₎ with high affinity.

Antibodies, antigen-binding fragments and methods for their generation are known in the art, for instance, as described in Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

Generally described, antibodies contain heavy chain polypeptides and light chain polypeptides. Antigen recognition is mediated by variable regions of the heavy chain and light chains. Complementarity determining region (CDR) refers to polypeptide regions within the variable region of heavy and light chains which form an antigen-binding site of an antibody. Three CDRs (CDR1, CDR2 and CDR3) are present in each light chain variable region (V_{L}) and each heavy chain variable region (V_{H}), i.e. Heavy Chain Variable Region CDRs: V_{H}CDR1, V_{H}CDR2, and V_{H}CDR3 and Light Chain Variable Region CDRs: V_{L}CDR1, V_{L}CDR2, and V_{L}CDR3. The CDRs are generally responsible for specific antigen recognition properties of the antibody.

Antibodies according to aspects of the present disclosure are mouse monoclonal anti-G4S₍ₙ₎ antibodies which specifically bind to G4S₍ₙ₎ with high affinity. Three representative anti-G4S₍ₙ₎ mouse mAbs are provided according to aspects of the present disclosure: Antibody A, Antibody B, and Antibody C.

All of Antibody A, Antibody B, and Antibody C specifically bind to G4S₍ₙ₎ with high affinity.

Hybridoma cells which produce Antibody A, or Antibody B, or Antibody C are provided according to aspects of the present disclosure, including hybridoma A which expresses mouse monoclonal antibody A; hybridoma B which expresses mouse monoclonal antibody B; and hybridoma C which expresses mouse monoclonal antibody C.

Antibodies according to aspects of the present disclosure are “isolated.” The term “isolated” in the context of a mouse monoclonal antibody refers to separation of the mouse monoclonal antibody from at least one other component present in the system in which the mouse monoclonal antibody was produced. For example, mouse monoclonal antibodies are separated from hybridoma cells, or the culture media, or body fluid in which they are produced, generating isolated mouse monoclonal antibodies.

According to aspects, mouse monoclonal antibodies are substantially purified to produce an isolated mouse monoclonal antibody. The term “substantially purified” refers to mouse monoclonal antibodies separated from other substances, such as those naturally present in a hybridoma cell, culture media, body fluid or chemical synthesis reaction, so that the mouse monoclonal antibodies make up at least about 0.01-100% of the mass, by weight, such as about 0.01%, 0.1%, 1%, 5%, 10%, 25%, 50% 75% or greater than about 75% of the mass, by weight, of the isolated mouse monoclonal antibodies.

Such purification is achieved by techniques illustratively including salt, pH, hydrophobic or affinity precipitation, electrophoretic methods such as gel electrophoresis and 2-D gel electrophoresis; chromatography methods such as HPLC, ion exchange chromatography, affinity chromatography, size exclusion chromatography, thin layer and paper chromatography.

Substitution at one or more amino acids in a CDR is possible while retaining specific antigen-binding function, particularly for those amino acid residues that do not contact the antigen. Such substitutions may be made at positions identified and known in the art or may be made empirically.

### Mouse Monoclonal Antibody mAb A (Antibody A)

Mouse monoclonal antibody mAb Antibody A (isotype IgG1, kappa), is characterized by:

V_{H}CDR1:
(SEQ ID NO: 1)
GFSLRNYGFG
V_{H}CDR2:
(SEQ ID NO: 2)
IWWNDNN
V_{H}CDR3:
(SEQ ID NO: 3)
TRIADDYHFDY
V_{L}CDR1:
(SEQ ID NO: 4)
QSLLNSRTRKNY
V_{L}CDR2:
(SEQ ID NO: 5)
WAS
V_{L}CDR3:
(SEQ ID NO: 6)
KQSYHLWT

Mouse monoclonal antibody mAb Antibody A (isotype IgG1, kappa), is further characterized by: a heavy chain variable region (VH, SEQ ID NO: 7) including framework regions and V_{H}CDR1, V_{H}CDR2, and V_{H}CDR3; and a light chain variable region (VL, SEQ ID NO: 8) including framework regions and V_{L}CDR1, V_{L}CDR2, and V_{L}CDR3.

SEQ ID NO: 7 Mouse monoclonal antibody
mAb A variable heavy chain
region (VH) (IgG2b) (119 amino acids):
QVTLKESGPGILQPSQTLSLTCSFSGFSLRNYGFGVGWMRQPSGK
GLEWLAHIWWNDNNYYNTALKSRLTISKDTSNNQVLLKIASVDSA
DTATYYCTRIADDYHFDYWGQGTTVIVSS

SEQ ID NO: 8 Mouse monoclonal antibody mAb A variable light chain region (kappa) (112 amino acids):

Light chain variable region
(LH, SEQ ID NO: 8) of mAb A:
DIVMSQSPSSLAVSTGEKVTMSCKSSQSLLNSRTRKNYLAWYQQK
PGQSPKLLIYWASTRESGVPDRFTGSGSGTDFTLTISTVQTEDLA
VYYCKQSYHLWTFGGGTKLEIK

According to aspects of the present disclosure, a mouse monoclonal antibody which is characterized by specific binding to G4S₍ₙ₎ includes a heavy chain variable region including the amino acid sequence SEQ ID NO: 7, or a variant thereof. The variant includes an amino acid sequence having at least 50%, at least 60% at least 70%, at least 75%, at least 80%, at least 85%, at least 90%, at least 95%, at least 96%, at least 97%, at least 98%, at least 99%, or greater than 99%, identity with the amino acid sequence of SEQ ID NO: 7 in framework regions and at least about 66%, 80%, 85%, 90%, 95%, 98%, 99% or more identity with CDR sequences therein, i.e., SEQ ID NOs: 1-3, wherein the variant retains at least a substantial proportion (at least about 50%, 60%, 70%, 80%, 90%, 95%, 98%, 99% or more) of the affinity/avidity and/or the specificity/selectivity of the parent antibody mAb A, and in some cases such an antibody may be associated with greater affinity, selectivity and/or specificity than the parent antibody mAb A.

According to aspects of the present disclosure, a mouse monoclonal antibody which is characterized by specific binding to G4S₍ₙ₎ includes a light chain variable region including the amino acid sequence SEQ ID NO: 8, or a variant thereof. The variant includes an amino acid sequence having at least 50%, at least 60% at least 70%, at least 75%, at least 80%, at least 85%, at least 90%, at least 95%, at least 96%, at least 97%, at least 98%, at least 99%, or greater than 99%, identity with the amino acid sequence of SEQ ID NO: 8 in framework regions and at least about 66%, 80%, 85%, 90%, 95%, 98%, 99% or more identity with CDR sequences therein, i.e., SEQ ID NOs: 4-6, wherein the variant retains at least a substantial proportion (at least about 50%, 60%, 70%, 80%, 90%, 95%, 98%, 99% or more) of the affinity/avidity and/or the specificity/selectivity of the parent antibody mAb A, and in some cases such an antibody may be associated with greater affinity, selectivity and/or specificity than the parent antibody mAb A.

### Mouse Monoclonal Antibody mAb B (Antibody B)

Mouse monoclonal antibody mAb B (isotype rat IgG2a, kappa), is characterized by:

V_{H}CDR1:
(SEQ ID NO: 9)
GFTENTAW
V_{H}CDR2:
(SEQ ID NO: 10)
MKAKSDSFAT
V_{H}CDR3:
(SEQ ID NO: 11)
AFGYGTSGDA
V_{L}CDR1:
(SEQ ID NO: 12)
QSLVGRSKNTY
V_{L}CDR2:
(SEQ ID NO: 13)
QVS
V_{L}CDR3:
(SEQ ID NO: 14)
LQGTHLPLT

Mouse monoclonal antibody mAb B (isotype rat IgG2a, kappa), is further characterized by: a heavy chain variable region (VH, SEQ ID NO: 15) including framework regions and V_{H}CDR1, V_{H}CDR2, and V_{H}CDR3; and a light chain variable region (VL, SEQ ID NO: 16) including framework regions and V_{L}CDR1, V_{L}CDR2, and V_{L}CDR3.

SEQ ID NO: 15 Mouse monoclonal antibody
mAb B variable heavy chain region (VH)
(119 amino acids):
EVQVLESGGGLVQPGSSLKLSCATSGFTFNTAWMYWYRQFPEKRL
EWVARMKAKSDSFATDYRESVKGRFTISRDDSKGSVYLHMYNVKE
EDTAIYYCAFGYGTSGDAWGQGASVTVSS
SEQ ID NO: 16 Mouse monoclonal antibody
mAb B variable light chain region
(112 amino acids):
DVVMTQTPPSLSVAIGQSVSISCKSSQSLVGRSKNTYLNWFLQSP
GRSPKRLIHQVSKLDSGVPDRFSGSGSERDFTLKISRVEAEDLGV
YYCLQGTHLPLTFGSGTKLEIK

According to aspects of the present disclosure, a mouse monoclonal antibody which is characterized by specific binding to G4S₍ₙ₎ includes a heavy chain variable region including the amino acid sequence SEQ ID NO: 15, or a variant thereof. The variant includes an amino acid sequence having at least 50%, at least 60% at least 70%, at least 75%, at least 80%, at least 85%, at least 90%, at least 95%, at least 96%, at least 97%, at least 98%, at least 99%, or greater than 99%, identity with the amino acid sequence of SEQ ID NO: 15 in framework regions and at least about 66%, 80%, 85%, 90%, 95%, 98%, 99% or more identity with CDR sequences therein, i.e., SEQ ID NOs: 9-11, wherein the variant retains at least a substantial proportion (at least about 50%, 60%, 70%, 80%, 90%, 95%, 98%, 99%, or greater than 99%) of the affinity/avidity and/or the specificity/selectivity of the parent antibody mAb B, and in some cases such an antibody may be associated with greater affinity, selectivity and/or specificity than the parent antibody mAb B.

According to aspects of the present disclosure, a mouse monoclonal antibody which is characterized by specific binding to G4S₍ₙ₎ includes a light chain variable region including the amino acid sequence SEQ ID NO: 16, or a variant thereof. The variant includes an amino acid sequence having at least 50%, at least 60% at least 70%, at least 75%, at least 80%, at least 85%, at least 90%, at least 95%, at least 96%, at least 97%, at least 98%, at least 99%, or greater than 99%, identity with the amino acid sequence of SEQ ID NO: 16 in framework regions and at least about 66%, 80%, 85%, 90%, 95%, 98%, 99% or more identity with CDR sequences therein, i.e., SEQ ID NOs: 12-14, wherein the variant retains at least a substantial proportion (at least about 50%, 60%, 70%, 80%, 90%, 95%, 98%, 99% or more) of the affinity/avidity and/or the specificity/selectivity of the parent antibody mAb B, and in some cases such an antibody may be associated with greater affinity, selectivity and/or specificity than the parent antibody mAb B.

Mouse monoclonal antibody mAb C (Antibody C) is identical in amino acid sequence to mAb B but is encoded by a different nucleotide sequence.

The phrase “specific binding” and grammatical equivalents as used herein in reference to binding of an anti-G4S₍ₙ₎ antibody, refers to binding of the mouse monoclonal antibody to G4S₍ₙ₎ without substantial binding to substances other than G4S₍ₙ₎. It is understood by the ordinarily skilled artisan that specific binding refers to specific binding as determinable by use of appropriate controls to distinguish it from nonspecific binding.

According to aspects of the present disclosure, the phrase “specific binding” and grammatical equivalents as used herein in reference to binding of an anti-G4S₍ₙ₎ antibody to G4S₍ₙ₎, without substantial binding to other substances. An anti-G4S₍ₙ₎ antibody according to aspects of the present disclosure specifically binds to G4S₍ₙ₎ when the antibody has an affinity constant (K_{A}) greater than 1×106 M for G4S₍ₙ₎.

Nucleic acid sequences disclosed herein may be used to produce recombinant antibodies and antigen binding fragments thereof. DNA encoding the antibody, or a portion thereof.

According to aspects of the present disclosure, one or more nucleic acid sequences disclosed herein is included in an expression cassette to produce recombinant antibodies and antigen binding fragments thereof. DNA encoding the antibody, or a portion thereof.

The term “expression cassette” is used herein to refer to a double-stranded recombinant DNA molecule containing a nucleic acid desired to be expressed and containing appropriate regulatory elements necessary or desirable for the transcription of the operably linked nucleic acid sequence in vitro or in vivo. The term “recombinant” is used to indicate a nucleic acid construct in which two or more nucleic acids are linked and which are not found linked in nature. The term “expressed” refers to transcription of a nucleic acid to produce a corresponding mRNA and/or translation of the mRNA to produce the corresponding protein. Expression cassettes can be generated recombinantly or synthetically or by DNA synthesis using well-known methodology.

An expression cassette is introduced into a cell using well-known methodology, such as, but not limited to, by introduction of a vector containing the expression cassette into the cell.

A “vector” is a nucleic acid that transfers an inserted nucleic acid into and/or between host cells becoming self-replicating. The term includes vectors that function primarily for insertion of a nucleic acid into a cell, replication of vectors that function primarily for the replication of nucleic acid, and expression vectors that function for transcription and/or translation of a nucleic acid. Also included are vectors that provide more than one of the above functions.

Vectors include plasmids, viruses, BACs, YACs, and the like. Particular viral vectors illustratively include those derived from adenovirus, adeno-associated virus and lentivirus.

The term “regulatory element” as used herein refers to a nucleotide sequence which controls some aspect of the expression of an operably linked nucleic acid. Exemplary regulatory elements illustratively include an enhancer, an internal ribosome entry site (IRES), an intron; an origin of replication, a polyadenylation signal (pA), a promoter, a transcription termination sequence, and an upstream regulatory domain, which contribute to the replication, transcription, post-transcriptional processing of a nucleic acid. Those of ordinary skill in the art are capable of selecting and using these and other regulatory elements in an expression construct with no more than routine experimentation.

The term “operably linked” as used herein refers to a nucleic acid in functional relationship with a second nucleic acid. The term “operably linked” encompasses functional connection of two or more nucleic acids, such as an oligonucleotide or polynucleotide to be transcribed and a regulatory element such as a promoter or an enhancer element, which allows transcription of the nucleic acid to be transcribed.

The term “promoter” as used herein refers to a DNA sequence operably linked to a nucleic acid to be transcribed such as a nucleic acid encoding a desired molecule. A promoter is generally positioned upstream of a nucleic acid sequence to be transcribed and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to SV40-pA, beta-globin-pA, the human growth hormone (hGH) pA and SCF-pA. The term “polyA” or “p (A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

Expression vectors are known in the art and include plasmids, cosmids, viruses, and bacteriophages, for example. Expression vectors can be prokaryotic vectors, insect vectors, or eukaryotic vectors, for example.

According to aspects of the present disclosure, an expression vector encoding SEQ ID NO:7 or a variant thereof, SEQ ID NO:8 or a variant thereof, both SEQ ID NOS: 7 and 8 or a variant of either or both thereof, are provided which can be used to express mouse monoclonal antibody mAb A or a portion thereof.

According to aspects of the present disclosure, an expression vector including SEQ ID NO:17 or a variant thereof, SEQ ID NO:18 or a variant thereof, both SEQ ID NOs: 17 and 18 or a variant of either or both thereof, are provided which can be used to express mouse monoclonal antibody mAb A or a portion thereof.

According to aspects of the present disclosure, an expression vector encoding SEQ ID NO:15 or a variant thereof, SEQ ID NO:16 or a variant thereof, both SEQ ID NOs: 15 and 16 or a variant of either or both thereof, are provided which can be used to express mouse monoclonal antibody mAb B or a portion thereof.

According to aspects of the present disclosure, an expression vector including SEQ ID NO: 19 or a variant thereof, SEQ ID NO:20 or a variant thereof, SEQ ID NO:21 or a variant thereof, SEQ ID NO:22 or a variant thereof, both SEQ ID NOs: 19 and 20 or a variant of either or both thereof, both SEQ ID NOs: 19 and 22 or a variant of either or both thereof, or both SEQ ID NOs: 20 and 21 or a variant of either or both thereof, are provided which can be used to express mouse monoclonal antibody mAb B or a portion thereof, C or a portion thereof, or a variant of mAb B or mAb C.

An expression vector can be introduced into a host cell for expression of the encoded proteins, and production of an antibody, or an antigen-binding fragment thereof, of the present disclosure. The host cell can be any cell capable of production of a protein from an encoding nucleic acid sequences, including prokaryotic or eukaryotic cells, such as, but not limited to, bacteria, yeast cells, fungi cells, plant cells, and animal cells, exemplified by insect cells and mammalian cells, e.g., murine cells, rat cells, macaque cells, or human cells, including cell lines such as but not limited to BHK cells; BRL 3A cells; CHO cells; COS-7 cells; HEK293 cells; HELA cells; HepG2 cells; MDCK cells; MMT cells; and MRC-5 cells; VERO-76 cells; and WI-38 cells.

Host cells containing the recombinant expression vector(s) are maintained under conditions wherein the desired protein is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

Methods and compositions of the present disclosure are not limited to particular amino acid sequences identified herein and variants of a reference peptide or protein are encompassed.

Methods and compositions of the present disclosure are not limited to particular nucleic acid sequences identified herein and variants of a reference nucleotide sequence are encompassed.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). The two sequences compared are generally the same length or nearly the same length. Optionally, the two sequences are natural variants of a structural domain of a protein or two related proteins.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length=12 to obtain nucleotide sequences homologous to a nucleic acid molecules of the present disclosure. BLAST protein searches are performed with the XBLAST program parameters set, e.g., to score 50, word length=3 to obtain amino acid sequences homologous to a protein molecule of the present disclosure. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleotide or amino acid mutations can be introduced without altering the functional properties of a given nucleic acid or protein, respectively. Mutations can be introduced using standard molecular biology techniques, such as site-directed mutagenesis and PCR-mediated mutagenesis, to produce variants. For example, one or more amino acid substitutions, additions, or deletions can be made without altering the functional properties of a reference protein.

When comparing a reference protein to a putative variant, amino acid similarity may be considered in addition to identity of amino acids at corresponding positions in an amino acid sequence. “Amino acid similarity” refers to amino acid identity and conservative amino acid substitutions in a putative variant compared to the corresponding amino acid positions in a reference protein.

Conservative amino acid substitutions can be made or may be present in reference proteins to produce or identify variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar/nonpolar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size; alanine, cysteine, aspartate, glycine, asparagine, proline, threonine, serine, valine are all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

According to aspects of the present disclosure, a mouse monoclonal antibody which is characterized by specific binding to G4S₍ₙ₎ includes a variable heavy chain region, or a variant thereof, encoded by the nucleotide sequence of SEQ ID NO: 17 or a variant of SEQ ID NO: 17 which hybridizes to the complement of SEQ ID NO: 17 under stringent hybridization conditions.

Mouse monoclonal antibody mAb A variable
heavy chain region (357 nucleotides)
SEQ ID NO: 17
CAGGTGACTCTCAAGGAGTCTGGCCCTGGCATTCTCCAACCCTCT
CAAACTTTGTCACTCACCTGTAGTTTTAGTGGTTTCAGTCTGAGG
AATTACGGTTTCGGGGTAGGCTGGATGAGGCAACCAAGCGGAAAA
GGGCTTGAGTGGTTGGCCCACATTTGGTGGAACGACAACAATTAC
TATAACACCGCCTTGAAAAGCCGCCTCACAATTTCCAAGGATACA
TCCAATAACCAAGTGCTGCTTAAAATTGCCAGCGTAGATTCTGCT
GACACTGCAACCTATTACTGTACCCGTATTGCCGACGACTATCAC
TTTGATTATTGGGGGCAAGGCACAACAGTCATAGTTAGCTCA

According to aspects of the present disclosure, a mouse monoclonal antibody which is characterized by specific binding to G4S₍ₙ₎ includes a variable light chain region, or a variant thereof, encoded by the nucleotide sequence of SEQ ID NO: 18 or a variant of SEQ ID NO: 18 which hybridizes to the complement of SEQ ID NO: 18 under stringent hybridization conditions.

Mouse monoclonal antibody mAb A variable
light chain region (336 nucleotides)
SEQ ID NO: 18
GATATAGTTATGTCACAGAGTCCATCCTCTCTCGCCGTGTCCACC
GGGGAAAAGGTGACTATGAGTTGTAAATCATCACAGAGTCTGTTG
AACTCTCGCACTCGCAAGAATTATCTCGCATGGTACCAGCAGAAA
CCAGGACAATCTCCAAAGCTGCTCATTTACTGGGCCAGTACACGG
GAGTCAGGTGTTCCAGACCGCTTCACTGGCAGTGGAAGTGGAACT
GATTTTACACTGACCATTAGCACAGTCCAGACTGAAGACTTGGCA
GTGTACTACTGCAAACAGAGCTATCACCTCTGGACTTTCGGCGGC
GGCACAAAACTTGAGATCAAA

According to aspects of the present disclosure, a mouse monoclonal antibody which is characterized by specific binding to G4S₍ₙ₎ includes: 1) a variable heavy chain region, or a variant thereof, encoded by the nucleotide sequence of SEQ ID NO: 17 or a variant of SEQ ID NO: 17 which hybridizes to the complement of SEQ ID NO: 17 under stringent hybridization conditions and 2) a variable light chain region, or a variant thereof, encoded by the nucleotide sequence of SEQ ID NO: 18 or a variant of SEQ ID NO: 18 which hybridizes to the complement of SEQ ID NO: 18 under stringent hybridization conditions.

According to aspects of the present disclosure, a mouse monoclonal antibody which is characterized by specific binding to G4S₍ₙ₎ includes a variable heavy chain region, or a variant thereof, encoded by the nucleotide sequence of SEQ ID NO: 19 or a variant of SEQ ID NO: 19 which hybridizes to the complement of SEQ ID NO: 19 under stringent hybridization conditions.

Mouse monoclonal antibody mAb B variable
heavy chain region (357 nucleotides)
SEQ ID NO: 19
GAAGTCCAAGTTCTCGAATCTGGGGGAGGTCTCGTACAACCAGGC
AGTTCATTGAAGCTTTCATGTGCTACTAGCGGGTTTACATTCAAT
ACCGCCTGGATGTACTGGTATCGTCAGTTTCCTGAAAAGAGGCTC
GAATGGGTAGCCCGAATGAAGGCCAAGTCCGATTCATTTGCCACT
GATTACAGGGAAAGCGTGAAGGGACGCTTCACTATCTCCCGTGAC
GACTCCAAGGGGTCAGTCTATCTCCACATGTATAACGTCAAAGAA
GAAGACACTGCAATTTACTACTGTGCATTCGGGTATGGAACTTCT
GGAGACGCCTGGGGCCAGGGTGCTTCTGTCACCGTGTCTAGC

According to aspects of the present disclosure, a mouse monoclonal antibody which is characterized by specific binding to G4S₍ₙ₎ includes a variable light chain region, or a variant thereof, encoded by the nucleotide sequence of SEQ ID NO: 20 or a variant of SEQ ID NO: 20 which hybridizes to the complement of SEQ ID NO: 20 under stringent hybridization conditions.

Mouse monoclonal antibody mAb B variable
light chain region (336 nucleotides)
SEQ ID NO: 20
GATGTGGTTATGACTCAGACCCCACCCAGTTTGAGTGTTGCTATC
GGCCAAAGTGTTAGTATATCATGCAAAAGCTCACAGTCCCTGGTA
GGGCGGTCAAAAAACACTTATCTTAACTGGTTTCTGCAGTCCCCA
GGTCGCAGCCCTAAAAGGCTTATACATCAAGTGTCTAAATTGGAT
AGCGGTGTTCCTGACCGCTTTAGTGGCAGTGGAAGCGAGCGAGAC
TTTACACTTAAAATAAGCCGTGTAGAAGCTGAAGACCTGGGCGTG
TATTATTGCCTCCAGGGGACTCATCTGCCTCTGACTTTCGGTTCT
GGTACAAAATTGGAAATTAAG

According to aspects of the present disclosure, a mouse monoclonal antibody which is characterized by specific binding to G4S₍ₙ₎ includes: 1) a variable heavy chain region, or a variant thereof, encoded by the nucleotide sequence of SEQ ID NO: 19 or a variant of SEQ ID NO: 19 which hybridizes to the complement of SEQ ID NO: 19 under stringent hybridization conditions and 2) a variable light chain region, or a variant thereof, encoded by the nucleotide sequence of SEQ ID NO: 20 or a variant of SEQ ID NO: 20 which hybridizes to the complement of SEQ ID NO: 20 under stringent hybridization conditions.

According to aspects of the present disclosure, a mouse monoclonal antibody which is characterized by specific binding to G4S₍ₙ₎ includes a variable heavy chain region, or a variant thereof, encoded by the nucleotide sequence of SEQ ID NO: 21 or a variant of SEQ ID NO: 21 which hybridizes to the complement of SEQ ID NO: 21 under stringent hybridization conditions.

Mouse monoclonal antibody mAb C variable
heavy chain region (357 nucleotides)
SEQ ID NO: 21
GAGGTTCAAGTCCTGGAAAGCGGTGGCGGGCTCGTGCAACCTGGT
TCCTCCCTTAAATTGAGTTGCGCCACTTCTGGATTTACTTTCAAC
ACAGCCTGGATGTATTGGTATAGGCAGTTCCCTGAAAAACGACTG
GAGTGGGTCGCAAGAATGAAAGCAAAGTCAGATTCCTTCGCCACT
GACTATCGAGAATCTGTCAAGGGTAGGTTCACAATAAGTCGCGAC
GATAGCAAGGGGTCAGTCTACCTGCATATGTATAACGTAAAAGAG
GAGGATACCGCCATCTATTACTGCGCTTTTGGTTATGGTACTAGC
GGAGACGCTTGGGGCCAGGGTGCTTCCGTCACTGTTAGTTCC

According to aspects of the present disclosure, a mouse monoclonal antibody which is characterized by specific binding to G4S₍ₙ₎ includes a variable light chain region, or a variant thereof, encoded by the nucleotide sequence of SEQ ID NO: 22 or a variant of SEQ ID NO: 22 which hybridizes to the complement of SEQ ID NO: 22 under stringent hybridization conditions.

Mouse monoclonal antibody mAb C variable
light chain region (336 nucleotides)
SEQ ID NO: 22
GACGTGGTTATGACTCAAACTCCCCCCTCTCTTTCCGTCGCCATA
GGTCAGAGCGTTAGCATTTCATGTAAGTCCTCACAGTCCTTGGTG
GGACGAAGCAAAAATACTTACCTCAACTGGTTTCTGCAAAGCCCA
GGGCGTTCTCCCAAGAGACTCATCCACCAGGTTTCTAAGCTGGAT
AGTGGAGTTCCAGATCGTTTTAGTGGTAGTGGGAGTGAGAGAGAT
TTTACACTCAAGATTTCTCGTGTGGAGGCAGAAGACCTCGGAGTA
TATTATTGCTTGCAAGGCACACATTTGCCACTGACCTTCGGGAGT
GGGACAAAGCTCGAAATCAAA

According to aspects of the present disclosure, a mouse monoclonal antibody which is characterized by specific binding to G4S₍ₙ₎ includes: 1) a variable heavy chain region, or a variant thereof, encoded by the nucleotide sequence of SEQ ID NO: 21 or a variant of SEQ ID NO: 21 which hybridizes to the complement of SEQ ID NO: 21 under stringent hybridization conditions and 2) a variable light chain region, or a variant thereof, encoded by the nucleotide sequence of SEQ ID NO: 22 or a variant of SEQ ID NO: 22 which hybridizes to the complement of SEQ ID NO: 22 under stringent hybridization conditions.

It will be appreciated by those of ordinary skill in the art that, due to the degenerate nature of the genetic code, alternate nucleic acid sequences encode the variable heavy chain regions, variable light chain regions, and variants thereof disclosed herein and that such alternate nucleic acids may be used in compositions and methods described herein.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002. A non-limiting example of high stringency hybridization conditions includes hybridization in 6×SSC, 5XDenhardt's solution, 30% formamide, and 100 micrograms/ml denatured salmon sperm at 37° C. overnight followed by washing in a solution of 0.1×SSC and 0.1% SDS at 60° C. for 15 minutes.

### Immunoassays

Detection of G4S₍ₙ₎ in a biological sample according to aspects of the present disclosure is accomplished by immunoassay using a mouse monoclonal antibody disclosed herein which specifically binds G4S₍ₙ₎. According to aspects of the present disclosure, an immunoassay is used to detect specific binding of a mouse monoclonal anti-G4S₍ₙ₎ antibody with G4S₍ₙ₎ in an expressed recombinant protein including a G4S₍ₙ₎ linker adjacent to a chimeric antigen receptor. According to aspects of the present disclosure, an immunoassay is used to detect specific binding of a mouse monoclonal anti-G4S₍ₙ₎ antibody with G4S₍ₙ₎ in an expressed recombinant protein including a G4S₍ₙ₎ linker adjacent to a chimeric antigen receptor in a T cell obtained from a subject.

The term “subject” as used herein can be a subject of any species in which CAR-T therapy is performed. According to particular aspects of the present disclosure, the subject is human.

Methods of detection of G4S₍ₙ₎ in a sample according to aspects of the present disclosure include contacting a sample containing or suspected of containing G4S₍ₙ₎ under antigen/antibody binding conditions with a detectably labeled mouse monoclonal anti-G4S₍ₙ₎ antibody of the present disclosure, wherein the detectable label is directly or indirectly attached to the mouse monoclonal anti-G4S₍ₙ₎ antibody. If present, the G4S₍ₙ₎ binds to the detectably labeled mouse monoclonal anti-G4S₍ₙ₎ antibody to form a complex such that specific detection of the complex is indicative of the G4S₍ₙ₎ in the sample, and where the G4S₍ₙ₎ is present in an expressed recombinant protein including a G4S₍ₙ₎ linker adjacent to a chimeric antigen receptor in a T cell obtained from a subject, the detection of G4S₍ₙ₎ is indicative of the presence of the adjacent CAR.

The term “sample” as used herein refers to any sample containing, or suspected of containing, G4S₍ₙ₎. According to aspects of the present disclosure, the sample contains, or is suspected of containing CAR-T cells which include an expression construct encoding a G4S₍ₙ₎ linker adjacent a CAR. According to aspects of the present disclosure, the sample contains, or is suspected of containing human CAR-T cells which include an expression construct encoding a G4S₍ₙ₎ linker adjacent a CAR.

The term “adjacent” as used herein refers to a G4S₍ₙ₎ linker expressed as part of a recombinant protein with a CAR. The G4S₍ₙ₎ may be immediately adjacent to the CAR or may be separated by 1-200 amino acids in the expressed recombinant protein. The G4S₍ₙ₎ may be adjacent to the N-terminus of the CAR or adjacent to the C-terminus of the CAR.

The term “detectable label” refers to any atom or moiety that can provide a detectable signal and which can be attached to a binding agent, such as a primary or secondary antibody or antigen binding fragment, or analyte. Examples of such detectable labels include fluorescent moieties, chemiluminescent moieties, bioluminescent moieties, ligands, particles, latex particles, luminescent particles, magnetic particles, fluorescent particles, colloidal gold, enzymes, enzyme substrates, radioisotopes, and chromophores. Such particles can be of any shape, size, composition, or physiochemical characteristics compatible with assay conditions. The particles can be microparticles having a diameter of less than one millimeter, for example, a size ranging from about 0.1 to about 1,000 micrometers in diameter, inclusive, such as about 3-25 microns in diameter, inclusive, or about 5-10 microns in diameter, inclusive. The particles can be nanoparticles having a diameter from about 1 nanometer (nm) to about 100,000 nm in diameter, inclusive, for example, a size ranging from about 10-1,000 nm, inclusive, or for example, a size ranging from 200-500 nm, inclusive.

Any appropriate method, including but not limited to spectroscopic, optical, photochemical, biochemical, enzymatic, electrical, isotopic, magnetic, energetic, and/or immunochemical method is used to detect a detectable label in a G4S₍ₙ₎ assay of the present disclosure.

Methods for attaching a detectable label to an antibody are well-known, including, for example, NHS (succinimidyl) ester methods, heterobifunctional methods (also called a “two-tag” method), a carbodiimide method, an isothiocyanate method, or a periodate method of conjugate of a detectable label to an antibody.

Immunoassays are well-known in the art and include, but are not limited to, enzyme-linked immunosorbent assay (ELISA) such as but not limited to, antigen capture ELISA, indirect ELISA, competitive ELISA, fixed cell ELISA; immunochromatography; antigen capture; flow cytometry; immunoblot; immunoprecipitation; immunodiffusion; competitive immunoassays, immunocytochemistry; radioimmunoassay; and combinations of any of these. Generalized details of immunoassays are described in standard references, illustratively including Wild, D., The Immunoassay Handbook, 3rd Ed., Elsevier Science, 2005; Gosling, J. P., Immunoassays: A Practical Approach, Practical Approach Series, Oxford University Press, 2005; E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; Ormerod, M. G., Flow Cytometry: a practical approach, Oxford University Press, 2000; and Givan, A. L., Flow Cytometry: first principles, Wiley, New York, 2001.

Immunoassay according to aspects of the present disclosure may include contacting an anti-G4S₍ₙ₎ antibody with a sample, wherein the anti-G4S₍ₙ₎ antibody is immobilized on a solid support to detect binding of the anti-G4S₍ₙ₎ antibody with G4S₍ₙ₎ in the biological sample.

Optionally an immunoassay according to aspects of the present disclosure is performed using a competitive immunoassay format including immobilization of a G4S₍ₙ₎ conjugate.

According to aspects of the present disclosure, an immunoassay to detect G4S₍ₙ₎ includes flow cytometry. Flow cytometry allows for rapid analysis one or more visible light properties and one or more fluorescence properties of single cells as the cells flow past one or more lasers.

The term “solid support” as used herein includes both solid supports and semi-solid supports. The term “solid porous support” as used herein includes both solid porous supports and semi-solid porous supports. The solid support can be in any of various forms or shapes, including planar, such as but not limited to membranes, silicon chips, glass plates and dipsticks; or three dimensional such as but not limited to particles, microtiter plates, microtiter wells, pins, and fibers.

A solid support for attachment of an antibody can be any of various materials such as glass; plastic, such as polypropylene, polystyrene, nylon; paper; silicon; nitrocellulose; or any other material to which the antibody, antigen binding fragment or antigen can be attached for use in an assay.

In particular aspects, a solid support to which an antibody, antigen binding fragment, or antigen is attached is a particle which is stable and insoluble under assay conditions. The particles can be of any shape, size, composition, or physiochemical characteristics compatible with assay conditions. The particle characteristics are optionally chosen so that the particle can be separated from fluid, e.g., on a filter with a particular pore size or by some other physical property, e.g., a magnetic property.

The particles are optionally latex particles, luminescent particles, magnetic particles, or fluorescent particles.

The particles can be of any shape, size, composition, or physiochemical characteristics compatible with assay conditions. The particles can be microparticles having a diameter of less than one millimeter, for example, a size ranging from about 0.1 to about 1,000 micrometers in diameter, inclusive, such as about 3-25 microns in diameter, inclusive, or about 5-10 microns in diameter, inclusive. The particles can be nanoparticles having a diameter from about 1 nanometer (nm) to about 100,000 nm in diameter, inclusive, for example, a size ranging from about 10-1,000 nm, inclusive, or for example, a size ranging from 200-500 nm, inclusive. The particles are can be organic or inorganic particles, such as glass or metal and can be particles of a synthetic or naturally occurring polymer, such as polystyrene, polycarbonate, silicon, nylon, cellulose, agarose, dextran, and polyacrylamide. Particles are latex beads according to aspects of the present disclosure.

Particles used are optionally encoded and distinguishable from other particles based on a characteristic such as color, reflective index and/or an imprinted or otherwise optically detectable pattern. For example, the particles may be encoded using optical, chemical, physical, or electronic tags. Encoded particles can contain or be attached to, one or more fluorophores which are distinguishable, for instance, by excitation and/or emission wavelength, emission intensity, excited state lifetime or a combination of these or other optical characteristics. Optical bar codes can be used to encode particles.

A solid support can be in the form of a lateral flow strip used in immunochromatography assay (ICA), i.e., a lateral flow test.

One or more washing steps is optionally included in an immunoassay according to aspects of the present disclosure, to remove unwanted and/or unbound materials.

Any reaction or diluent buffer compatible with the sample, reagents and reaction can be used in immunoassays, such as for dilution and/or washing steps, including but not limited to phosphate buffered saline, sodium phosphate buffer, potassium phosphate buffer, Tris-HCl buffer, Tricine buffer and other buffers described herein.

The sample may be diluted or processed to purify or concentrate G4S₍ₙ₎ prior to analysis. G4S₍ₙ₎ contained in a sample is optionally purified or concentrated for assay according to a method of the present disclosure.

The term “purified” in the context of a biological sample refers to separation of G4S₍ₙ₎ in the sample from at least one other component present in the biological sample. According to aspects of the present disclosure, the term “purified” refers to purification of a G4S₍ₙ₎ linker adjacent to a CAR in a recombinant protein construct i.e. the CAR is not separated from the adjacent G4S₍ₙ₎ linker.

According to aspects, a G4S₍ₙ₎ linker adjacent to a CAR in a recombinant protein construct is substantially purified from the biological sample to produce a substantially purified sample for use in an inventive assay. The term “substantially purified” refers to a desired material separated from other substances naturally present in a sample obtained from the subject so that the desired material makes up at least about 0.01-100% of the mass, by weight, such as about 0.01%, 0.1%, 1%, 5%, 10%, 25%, 50% 75% or greater than about 75% of the mass, by weight, of the substantially purified sample.

Sample purification is achieved by techniques illustratively including salt, pH, hydrophobic or affinity precipitation, electrophoretic methods such as gel electrophoresis and 2-D gel electrophoresis; chromatography methods such as HPLC, ion exchange chromatography, affinity chromatography, size exclusion chromatography, thin layer and paper chromatography. It is appreciated that electrophoresis and chromatographic methods can also be used to separate a peptide or peptides from other components in a sample in the course of performing an assay, as in, for example separation of proteins in immunoblot assays.

According to one aspect of the present disclosure, a G4S₍ₙ₎ linker adjacent to a CAR in a recombinant protein construct is isolated and concentrated by absorption onto a solid substrate.

According to aspects of the present disclosure, immunoassay includes assay of a G4S₍ₙ₎ linker adjacent to a CAR in a recombinant protein construct in a sample by an ELISA technique.

Optionally, a control or standard is included in an assay according to aspects of the present disclosure.

The terms “control” and “standard” are familiar to those of ordinary skill in the art and refer to any control or standard that can be used for comparison. The control or standard may be determined prior to assay for a G4S₍ₙ₎ linker adjacent to a CAR in a recombinant protein construct, in parallel, simultaneously, in a multiplex assay or other assay format. A control or standard can be a negative control and/or a positive control.

A sample which is assayed for a G4S₍ₙ₎ linker adjacent to a CAR in a recombinant protein construct according to methods of the disclosure may be any sample containing or suspected of containing a G4S₍ₙ₎ linker adjacent to a CAR in a recombinant protein construct including, an aqueous buffer, a mammalian cell culture medium, a bacterial culture medium, cell or bacterial extract, whole blood, plasma, serum, urine, saliva, and other human or animal body fluids.

According to aspects of the present disclosure, the sample which is assayed for a G4S₍ₙ₎ linker adjacent to a CAR in a recombinant protein construct includes intact cells in which the G4S₍ₙ₎ linker portion of the construct is detectable on the surface of the intact cells using an immunoassay including an anti-G4S₍ₙ₎ antibody of the present disclosure.

According to aspects of the present disclosure, the sample which is assayed for a G4S₍ₙ₎ linker adjacent to a CAR in a recombinant protein construct includes intact cells in which the G4S₍ₙ₎ linker portion of the construct is detectable on the surface of the intact cells using a flow cytometry immunoassay including an anti-G4S₍ₙ₎ antibody of the present disclosure

According to aspects, immunoassay kits for detecting G4S₍ₙ₎ linker adjacent to a CAR in a recombinant protein construct in a biological sample are provided which include one or more antibodies which specifically bind to G4S₍ₙ₎.

According to aspects, immunoassay kits for detecting G4S₍ₙ₎ linker adjacent to a CAR in a recombinant protein construct in a biological sample are provided which include one or both of: mAb B and mAb A, an antigen binding fragment which specifically binds to G4S₍ₙ₎, or a variant of any thereof which specifically binds to G4S₍ₙ₎.

One or more auxiliary components are optionally included in such kits, such as one or more of: a control, a secondary antibody, one or more reaction vessels, a buffer, diluent, and a reconstituting agent.

Embodiments of inventive compositions and methods are illustrated in the following examples. These examples are provided for illustrative purposes and are not considered limitations on the scope of inventive compositions and methods.

### EXAMPLES

### Generation and Validation of Mouse Monoclonal Anti-G4S₍ₙ₎ Antibodies

To generate mouse monoclonal antibodies of the present disclosure which specifically bind to G4S₍ₙ₎ linkers, BALB/c mice were immunized with the peptide KLH-GGGGSGGGGSGGGGS (SEQ ID NO:23), i.e. Keyhole Limpet Hemocyanin-G4S₍₃₎ every 2 weeks for a total of 5 immunizations. A week following the 5th immunization, a mandibular bleed was done and the serum tested by Flow Cytometry. A 6^{th} immunization was administered and a week later the serum was tested again by Flow Cytometry. The best responding mouse was chosen and a fusion boost was done 3 days prior to the fusion.

Hybridomas were formed using standard protocols to fuse myeloma cells with spleen cells, and lymph node cells were drained and harvested. Successful hybridomas were selected into HAT medium (hypoxanthine-aminopterin-thymidine medium), and approximately one cell was deposited per well in microtiter plates for further evaluation.

Cell culture supernatants from these microtiter plates were tested by Flow Cytometry against two types of G4S₍ₙ₎-expressing cell transfectants: 1) CHO cells transfected with a G4S₍ₙ₎ linker, and 2) K562 cells transfected with a G4S₍ₙ₎ linker. Supernatants with the highest specific antibody production were selected by assessment of staining profiles and then sub-cultured into larger vessels and sub-cloned. The CHO transfectant was generated by transfection with a vector including a G4S₍ₙ₎ linker insert sequences: HA-GGGS-H-(G4S) 4-L-GGGS-TM (SEQ ID NO:24), H-(G4S) 4-L-GGSGSAGSAGSGEF-HA-GGGS-TM (SEQ ID NO:25), and HA-GGGS-H-(G4S) 4-L-GGGS-TM (SEQ ID NO:26). In these G4S₍ₙ₎ linker insert sequences, “HA” is a hemagglutinin epitope YPYDVPDYA (HA.11, SEQ ID NO:27) The K562 transfectant was generated using a pLVIP-GFP vector including a G4S₍ₙ₎ linker insert: GGGGSGGGGSGGGGS (SEQ ID NO:28) Polyethyleneimine (PEI) chemical transfection reagent was used to deliver the vector to the nucleus of the cells.

The transfected cells were grown in RPMI media supplemented with 10% FBS in T75 culture flask and then tested for viability following several days of growth.

Viable transfected cells were co-incubated with two types of distinguishably labeled antibodies: 1) mouse monoclonal anti-G4S₍ₙ₎ linker antibodies of the present disclosure; and 2) an anti-HA.11 antibody which specifically recognizes the influenza hemagglutinin epitope (YPYDVPDYA, SEQ ID NO:27) included in the G4S₍ₙ₎ linker expression vectors. Alternatively, the viable transfected cells were co-incubated with distinguishably labeled: 1) isotype control antibody of the present disclosure; and 2) an anti-HA.11 antibody which specifically recognizes the influenza hemagglutinin epitope (YPYDVPDYA, SEQ ID NO:27) included in the G4S₍ₙ₎ linker expression vectors.

In this example, 0.5 ug, 1.0 ug, or 2.0 ug of the mouse monoclonal anti-G4S₍ₙ₎ linker antibodies of the present disclosure, or the isotype control antibodies, were used.

Isotype controls used were RTK2758 (Rat IgG2a, κ); MPC-11 (Mouse IgG2b, κ); and MOPC-21 (Mouse IgG1, κ).

The combination of either: 1) a distinguishably labeled mouse monoclonal anti-G4S₍ₙ₎ linker antibody of the present disclosure; and a distinguishably labeled anti-HA.11 antibody; or 2) a distinguishably labeled isotype control; and 2) a distinguishably labeled anti-HA.11 antibody, were incubated together with the transfected cells in immunostaining buffer for 15 minutes at room temperature in the dark. After this incubation, the cells were washed with the staining buffer and then centrifuged at 1200 rpm for five minutes. This washing step was repeated one more time and then the stained cells were resuspended in 300-500 μL of staining buffer. Ten minutes prior to flow cytometric data acquisition, 10 μL of live/dead solution, e.g. propidium iodide, was added to the samples for viability staining. Data were then acquired on a commercially available Flow Cytometer and post data-acquisition analysis was performed using commercially available software (FlowJo). All the data were analyzed and presented in the dot plot format which generally utilized for multiparameter analysis.

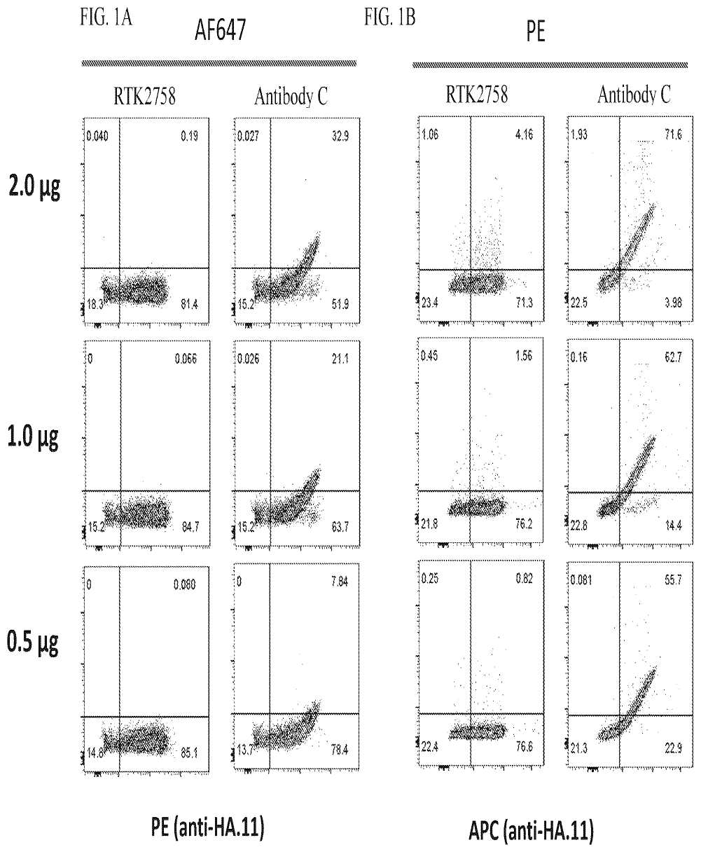

FIGS. 1A and 1B show graphs representing results of flow cytometry immunoassays assessing binding of mouse monoclonal anti-G4S₍ₙ₎ linker antibody C in amounts of 0.5 ug, 1.0 ug, and 2.0 ug compared to the same amounts of an isotype control (RTK2758) on G4S₍ₙ₎ linker-transfected CHO cells. Antibody C or isotype control antibody RTK2758 were directly conjugated to either AF647 (FIG. 1A) or PE (FIG. 1B). The co-stain, anti-HA.11 antibody, was directly labeled with either PE (FIG. 1A) or allophycocyanin (APC). Detected fluorescent signal from the various amounts of Antibody C or isotype control antibody are shown on the y-axis and detected signal from the distinguishably labeled anti-HA epitope HA.11 antibody is shown on the x-axis in each graph. (FIG. 1B).

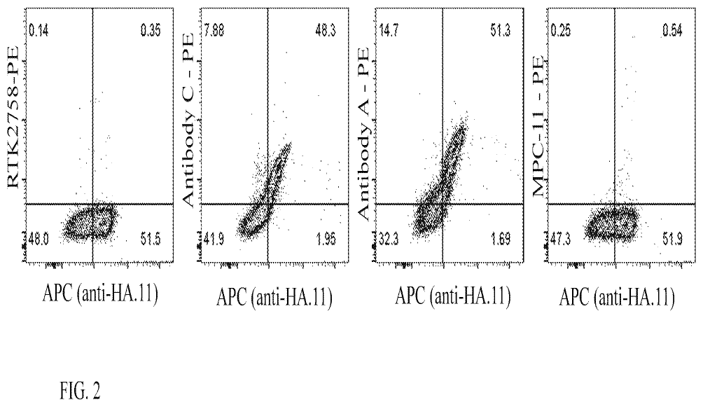

FIG. 2 shows graphs representing results of flow cytometry assays assessing co-staining of mouse monoclonal anti-G4S₍ₙ₎ linker antibody C or A with anti-HA.11 antibody; or isotype control antibody RTK2758 or MPC-11, with anti-HA.11 antibody; on G4S₍ₙ₎ linker-transfected CHO cells. Anti-G4S₍ₙ₎ linker antibodies C and A, and isotype control antibodies RTK2758 and MPC-11 were all directly conjugated to PE and the detected fluorescent signal is shown on the y-axis. The anti-HA.11 antibody was directly labeled to APC and detected fluorescent signal from the anti-HA.11 antibody used to co-stain the G4S₍ₙ₎ linker-transfected CHO cells is shown on the x-axis.

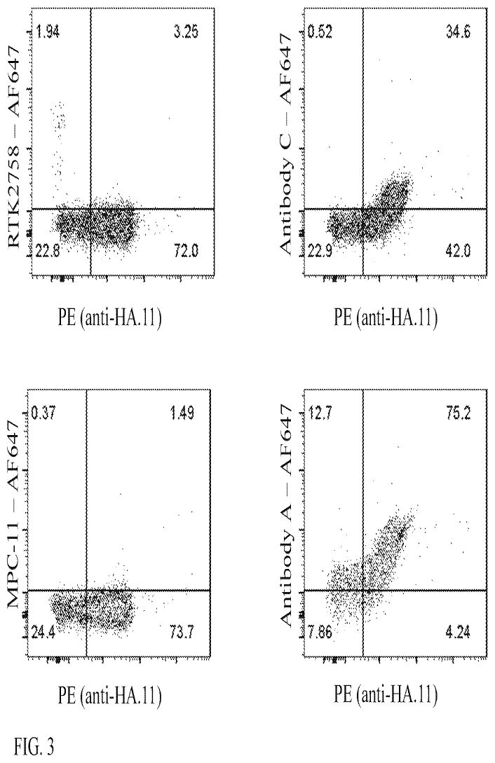

FIG. 3 shows graphs representing results of flow cytometry assays assessing co-staining of mouse monoclonal anti-G4S₍ₙ₎ linker antibody C or A with anti-HA.11 antibody; or isotype control antibody RTK2758 or MPC-11, with anti-HA.11 antibody; on G4S₍ₙ₎ linker-transfected CHO cells. Anti-G4S₍ₙ₎ linker antibodies C and A, and isotype control antibodies RTK2758 and MPC-11 were all directly conjugated to AF647 and the detected fluorescent signal is shown on the y-axis. The anti-HA.11 antibody was directly conjugated to PE and detected fluorescent signal from the anti-HA.11 antibody used to co-stain the G4S₍ₙ₎ linker-transfected CHO cells is shown on the x-axis.

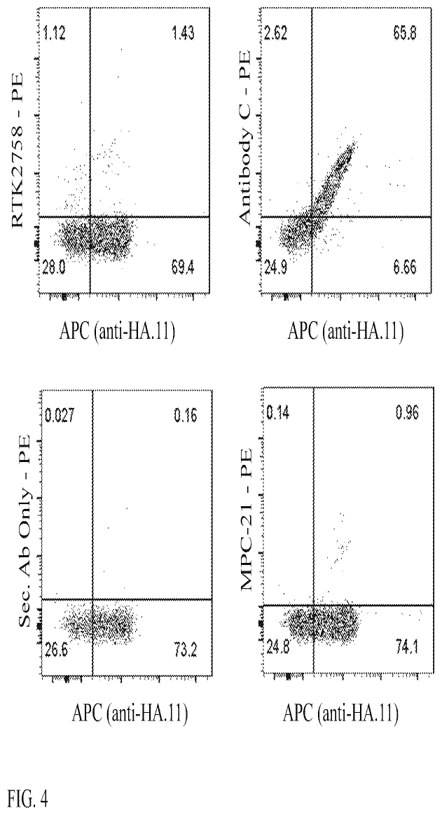

FIG. 4 shows graphs representing results of flow cytometry assays assessing co-staining of mouse monoclonal anti-G4S₍ₙ₎ linker antibody C with anti-HA.11 antibody; or isotype control antibody RTK2758 or MPC-21, with anti-HA.11 antibody; on G4S₍ₙ₎ linker-transfected CHO cells. A secondary antibody alone (i.e. with no primary antibody present) directly conjugated to PE, was incubated with the transfected CHO cells to assess background non-specific fluorescence and the detected fluorescent signal is shown on the y-axis. Anti-G4S₍ₙ₎ linker antibody C, and isotype control antibodies RTK2758 and MPC-21 were all directly conjugated to PE and the detected fluorescent signal is shown on the y-axis. The anti-HA.11 antibody was directly conjugated to APC and detected fluorescent signal from the anti-HA.11 antibody used to co-stain the G4S₍ₙ₎ linker-transfected CHO cells is shown on the x-axis.

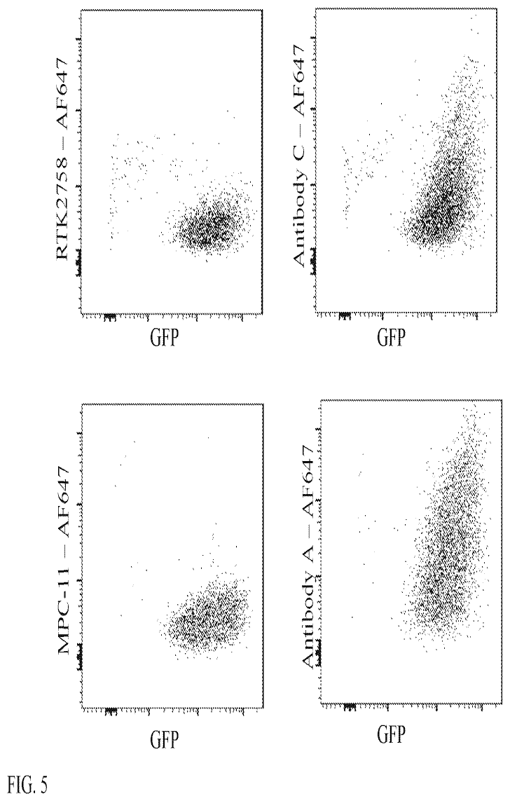

FIG. 5 shows results of flow cytometry assays assessing immunostaining of K562 cells transfected with an expression construct encoding a G4S₍ₙ₎ linker and green fluorescent protein (GFP). The transfected K562 cells were incubated with mouse monoclonal anti-G4S₍ₙ₎ linker antibody C or A; or isotype control antibody RTK2758 or MPC-11. Anti-G4S₍ₙ₎ linker antibodies C and A, and isotype control antibodies RTK2758 and MPC-11 were all directly conjugated to AF647 and the detected fluorescent signal is shown on the y-axis. Detected GFP signal, indicative of expression of the expression construct encoding the G4S₍ₙ₎ linker and GFP, is shown on the x-axis.

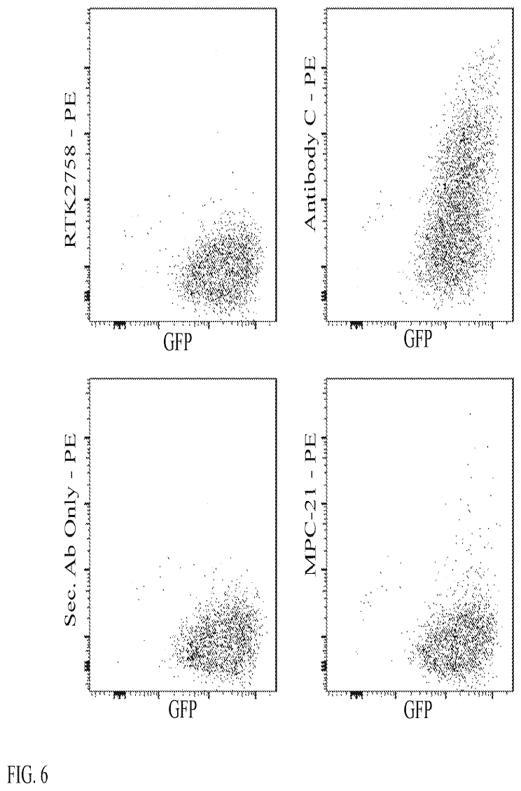

FIG. 6 shows results of flow cytometry assays assessing immunostaining of K562 cells transfected with an expression construct encoding a G4S₍ₙ₎ linker and green fluorescent protein (GFP). The transfected K562 cells were incubated with mouse monoclonal anti-G4S₍ₙ₎ linker antibody C; or isotype control antibody RTK2758 or MPC-21. A secondary antibody alone (i.e. with no primary antibody present) directly conjugated to PE, was incubated with the transfected K562 cells to assess background non-specific fluorescence and the detected fluorescent signal is shown on the y-axis. Anti-G4S₍ₙ₎ linker antibody C, and isotype control antibodies RTK2758 and MPC-21 were all directly conjugated to PE and the detected fluorescent signal is shown on the y-axis. Detected GFP signal, indicative of expression of the expression construct encoding the G4S₍ₙ₎ linker and GFP, is shown on the x-axis.

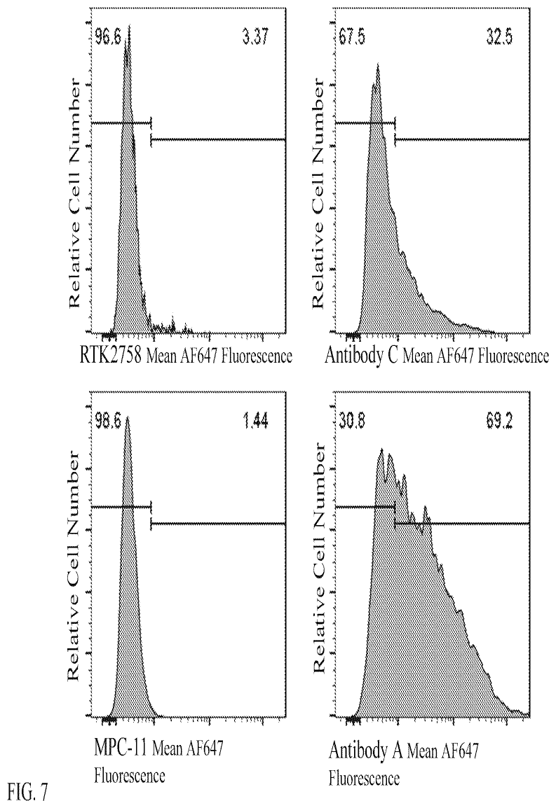

FIG. 7 shows histograms representing single parameter analysis of results of flow cytometry assays assessing immunostaining of mouse monoclonal anti-G4S₍ₙ₎ linker antibody C or A; or isotype control antibody RTK2758 or MPC-11; on G4S₍ₙ₎ linker-transfected K562 cells. Anti-G4S₍ₙ₎ linker antibodies C and A, and isotype control antibodies RTK2758 and MPC-11, were all directly conjugated to AF647 and mean AF647 fluorescence is shown on the y-axis. Relative cell number is shown on the x-axis.

### Item List

Item 1. A mouse monoclonal antibody which specifically binds to G4S₍ₙ₎.
Item 2. The mouse monoclonal antibody of item 1, comprising heavy chain variable region complementarity determining regions (V_{H}CDR): V_{H}CDR1 of SEQ ID NO: 1, V_{H}CDR2 of SEQ ID NO:2, and V_{H}CDR3 of SEQ ID NO:3, or a variant of any one, two or three of SEQ ID NO: 1, SEQ ID NO:2, or SEQ ID NO:3.
Item 3. The mouse monoclonal antibody of item 1 or item 2, comprising light chain variable region complementarity determining regions (V_{L}CDR): V_{L}CDR1 of SEQ ID NO: 4, V_{L}CDR2 of SEQ ID NO:5, and V_{L}CDR3 of SEQ ID NO:6, or a variant of any one, two, or three SEQ ID NO: 4, SEQ ID NO:5, or SEQ ID NO:6.
Item 4. The mouse monoclonal antibody of any one of items 1 to 3, comprising a heavy chain variable region (V_{H}) of SEQ ID NO: 7, or a variant thereof.
Item 5. The mouse monoclonal antibody of any one of items 1 to 4, comprising a light chain variable region (V_{L}) of SEQ ID NO: 8, or a variant thereof.
Item 6. The mouse monoclonal antibody of any one of items 1 to 5, comprising a heavy chain variable region (V_{H}) encoded by SEQ ID NO:17, or a variant thereof.
Item 7. The mouse monoclonal antibody of any one of items 1 to 6, comprising a light chain variable region (V_{L}) encoded by SEQ ID NO:18, or a variant thereof.
Item 8. The mouse monoclonal antibody of item 1, comprising heavy chain variable region complementarity determining regions (V_{H}CDR): V_{H}CDR1 of SEQ ID NO: 9, V_{H}CDR2 of SEQ ID NO:10, and V_{H}CDR3 of SEQ ID NO:11, or a variant of any one, two or three of SEQ ID NO: 9, SEQ ID NO: 10, or SEQ ID NO:11.
Item 9. The mouse monoclonal antibody of item 1 or item 8, comprising light chain variable region complementarity determining regions (V_{L}CDR): V_{L}CDR1 of SEQ ID NO: 12, V_{L}CDR2 of SEQ ID NO:13, and V_{L}CDR3 of SEQ ID NO:14, or a variant of any one, two, or three SEQ ID NO: 12, SEQ ID NO: 13, or SEQ ID NO14.
Item 10. The mouse monoclonal antibody of any one of items 1, 8, or 9, comprising a heavy chain variable region (V_{H}) of SEQ ID NO: 15, or a variant thereof.
Item 11. The mouse monoclonal antibody of any one of items 1, or 8 to 10, comprising a light chain variable region (V_{L}) of SEQ ID NO: 16, or a variant thereof.
Item 12. The mouse monoclonal antibody of any one of items 1, or 8 to 11, comprising a heavy chain variable region (V_{H}) encoded by SEQ ID NO:19, SEQ ID NO: 21, or a variant or either thereof.
Item 13. The mouse monoclonal antibody of any one of items 1, or 8 to 12, comprising a light chain variable region (V_{L}) encoded by SEQ ID NO:20, SEQ ID NO:22, or a variant of either thereof.
Item 14. The mouse monoclonal antibody of any one of items 1 to 7, comprising a variant of heavy chain variable region 1 encoded by a nucleotide sequence which hybridizes to the complement of SEQ ID NO: 17, under stringent hybridization conditions.
Item 15. The mouse monoclonal antibody of any one of items 1 to 7 or 14, comprising a variant of light chain variable region 1 encoded by a nucleotide sequence which hybridizes to the complement of SEQ ID NO: 18, under stringent hybridization conditions.
Item 16. The mouse monoclonal antibody of any one of items 8 to 13, comprising a variant of heavy chain variable region 1 encoded by a nucleotide sequence which hybridizes to the complement of SEQ ID NO: 19, or SEQ ID NO: 21, under stringent hybridization conditions.
Item 17. The mouse monoclonal antibody of any one of items 8 to 13 or 16, comprising a variant of light chain variable region 1 encoded by a nucleotide sequence which hybridizes to the complement of SEQ ID NO: 20, or SEQ ID NO: 22, under stringent hybridization conditions.
Item 18. The mouse monoclonal antibody of any one of items 1 to 17, further comprising a detectable label.
Item 19. An expression cassette comprising a nucleic acid encoding a mouse monoclonal antibody of any one of items 1 to 18.
Item 20. An expression vector comprising an expression cassette according to item 19.
Item 21. A host cell comprising an expression vector encoding a mouse monoclonal antibody of any one of items 1 to 18.
Item 22. A method for detecting G4S₍ₙ₎ in a sample, comprising: contacting a sample containing or suspected of containing G4S₍ₙ₎ with a mouse monoclonal anti-G4S₍ₙ₎ antibody of any one of items 1 to 18, under binding conditions; and detecting binding of the anti-G4S₍ₙ₎ antibody thereof with G4S₍ₙ₎.
Item 23. The method of item 22, wherein the method comprises an ELISA.
Item 24. The method of item 22 or item 23, wherein the method comprises immunochromatography; antigen capture; flow cytometry; immunoblot; immunofluorescence; immunoprecipitation; immunodiffusion; competitive immunoassay, immunocytochemistry; radioimmunoassay; and combinations of any of these.
Item 25. The method of any one of items 22 to 24, wherein the sample comprises CAR-T cells, wherein the CAR-T cells comprise an expression construct encoding a G4S₍ₙ₎ adjacent to a chimeric antigen receptor.
Item 26. The method of item 25, wherein the sample comprises human CAR-T cells.
Item 27. An immunoassay kit, comprising: one or more antibodies of any one of items 1 to 18, and/or a host cell of item 21.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A mouse monoclonal antibody which specifically binds to G4S₍ₙ₎.

2. The mouse monoclonal antibody of claim 1, comprising heavy chain variable region complementarity determining regions (V_{H}CDR): V_{H}CDR1 of SEQ ID NO: 1, V_{H}CDR2 of SEQ ID NO:2, and V_{H}CDR3 of SEQ ID NO:3, or a variant of any one, two or three of SEQ ID NO: 1, SEQ ID NO:2, or SEQ ID NO:3.

3. The mouse monoclonal antibody of claim 1, comprising light chain variable region complementarity determining regions (V_{L}CDR): V_{L}CDR1 of SEQ ID NO: 4, V_{L}CDR2 of SEQ ID NO:5, and V_{L}CDR3 of SEQ ID NO:6, or a variant of any one, two, or three SEQ ID NO: 4, SEQ ID NO:5, or SEQ ID NO:6.

4. The mouse monoclonal antibody of claim 1, comprising a heavy chain variable region (V_{H}) of SEQ ID NO: 7, or a variant thereof.

5. The mouse monoclonal antibody of claim 1, comprising a light chain variable region (V_{L}) of SEQ ID NO: 8, or a variant thereof.

6. The mouse monoclonal antibody of claim 1, comprising a heavy chain variable region (V_{H}) encoded by SEQ ID NO:17, or a variant thereof.

7. The mouse monoclonal antibody of claim 1, comprising a light chain variable region (V_{L}) encoded by SEQ ID NO: 18, or a variant thereof.

8. The mouse monoclonal antibody of claim 1, comprising heavy chain variable region complementarity determining regions (V_{H}CDR): V_{H}CDR1 of SEQ ID NO: 9, V_{H}CDR2 of SEQ ID NO: 10, and V_{H}CDR3 of SEQ ID NO:11, or a variant of any one, two or three of SEQ ID NO: 9, SEQ ID NO:10, or SEQ ID NO:11.

9. The mouse monoclonal antibody of claim 1, comprising light chain variable region complementarity determining regions (V_{L}CDR): V_{L}CDR1 of SEQ ID NO: 12, V_{L}CDR2 of SEQ ID NO:13, and V_{L}CDR3 of SEQ ID NO:14, or a variant of any one, two, or three SEQ ID NO: 12, SEQ ID NO:13, or SEQ ID NO14.

10. The mouse monoclonal antibody of claim 1, comprising a heavy chain variable region (V_{H}) of SEQ ID NO: 15, or a variant thereof.

11. The mouse monoclonal antibody of claim 1, comprising a light chain variable region (V_{L}) of SEQ ID NO: 16, or a variant thereof.

12. The mouse monoclonal antibody of claim 1, comprising a heavy chain variable region (V_{H}) encoded by SEQ ID NO:19, SEQ ID NO:21, or a variant or either thereof.

13. The mouse monoclonal antibody of claim 1, comprising a light chain variable region (V_{L}) encoded by SEQ ID NO:20, SEQ ID NO:22, or a variant of either thereof.

14. The mouse monoclonal antibody of claim 1, comprising a variant of heavy chain variable region 1 encoded by a nucleotide sequence which hybridizes to the complement of SEQ ID NO: 17, under stringent hybridization conditions.

15. The mouse monoclonal antibody of claim 1, comprising a variant of light chain variable region 1 encoded by a nucleotide sequence which hybridizes to the complement of SEQ ID NO: 18, under stringent hybridization conditions.

16. The mouse monoclonal antibody of claim 8, comprising a variant of heavy chain variable region 1 encoded by a nucleotide sequence which hybridizes to the complement of SEQ ID NO: 19, or SEQ ID NO: 21, under stringent hybridization conditions.

17. The mouse monoclonal antibody of claim 8, comprising a variant of light chain variable region 1 encoded by a nucleotide sequence which hybridizes to the complement of SEQ ID NO: 20, or SEQ ID NO: 22, under stringent hybridization conditions.

18. The mouse monoclonal antibody of claim 1, further comprising a detectable label.

19. An expression cassette comprising a nucleic acid encoding a mouse monoclonal antibody of claim 1.

20. An expression vector comprising an expression cassette according to claim 19.

21. A host cell comprising an expression vector encoding a mouse monoclonal antibody of claim 1.

22. A method for detecting G4S₍ₙ₎ in a sample, comprising:
contacting a sample containing or suspected of containing G4S₍ₙ₎ with a mouse monoclonal anti-G4S₍ₙ₎ antibody of claim 1, under binding conditions; and detecting binding of the anti-G4S₍ₙ₎ antibody thereof with G4S₍ₙ₎.

23. The method of claim 22, wherein the method comprises an ELISA.

24. The method of claim 22, wherein the method comprises immunochromatography; antigen capture; flow cytometry; immunoblot; immunofluorescence; immunoprecipitation; immunodiffusion; competitive immunoassay, immunocytochemistry; radioimmunoassay; and combinations of any of these.

25. The method of claim 22, wherein the sample comprises CAR-T cells, wherein the CAR-T cells comprise an expression construct encoding a G4S₍ₙ₎ adjacent to a chimeric antigen receptor.

26. The method of claim 25, wherein the sample comprises human CAR-T cells.

27. An immunoassay kit, comprising: one or more antibodies of claim 1.


SEQUENCE LISTING (32 sequences)

SEQ ID NO: 1 - protein, 10 aa, synthetic construct
GFSLRNYGFG

SEQ ID NO: 2 - protein, 7 aa, synthetic construct
IWWNDNN

SEQ ID NO: 3 - protein, 11 aa, synthetic construct
TRIADDYHFDY

SEQ ID NO: 4 - protein, 12 aa, synthetic construct
QSLLNSRTRKNY

SEQ ID NO: 5 - skipped

SEQ ID NO: 6 - protein, 8 aa, synthetic construct
KQSYHLWT

SEQ ID NO: 7 - protein, 119 aa, synthetic construct
QVTLKESGPGILQPSQTLSLTCSFSGFSLRNYGFGVGWMRQPSGKGLEWLAHIWWNDNNYYNTALKSRLTISKDTSNNQVLLKIASVDSADTATYYCTRIADDYHFDYWGQGTTVIVSS

SEQ ID NO: 8 - protein, 112 aa, synthetic construct
DIVMSQSPSSLAVSTGEKVTMSCKSSQSLLNSRTRKNYLAWYQQKPGQSPKLLIYWASTRESGVPDRFTGSGSGTDFTLTISTVQTEDLAVYYCKQSYHLWTFGGGTKLEIK

SEQ ID NO: 9 - protein, 8 aa, synthetic construct
GFTFNTAW

SEQ ID NO: 10 - protein, 10 aa, synthetic construct
MKAKSDSFAT

SEQ ID NO: 11 - protein, 10 aa, synthetic construct
AFGYGTSGDA

SEQ ID NO: 12 - protein, 11 aa, synthetic construct
QSLVGRSKNTY

SEQ ID NO: 13 - skipped

SEQ ID NO: 14 - protein, 9 aa, synthetic construct
LQGTHLPLT

SEQ ID NO: 15 - protein, 119 aa, synthetic construct
EVQVLESGGGLVQPGSSLKLSCATSGFTFNTAWMYWYRQFPEKRLEWVARMKAKSDSFATDYRESVKGRFTISRDDSKGSVYLHMYNVKEEDTAIYYCAFGYGTSGDAWGQGASVTVSS

SEQ ID NO: 16 - protein, 112 aa, synthetic construct
DVVMTQTPPSLSVAIGQSVSISCKSSQSLVGRSKNTYLNWFLQSPGRSPKRLIHQVSKLDSGVPDRFSGSGSERDFTLKISRVEAEDLGVYYCLQGTHLPLTFGSGTKLEIK

SEQ ID NO: 17 - DNA, 357 bp, synthetic construct
  source 1..357: mol_type = other DNA
caggtgactctcaaggagtctggccctggcattctccaaccctctcaaactttgtcactcacctgtagttttagtggtttcagtctgaggaattacggtttcggggtaggctggatgaggcaaccaagcggaaaagggcttgagtggttggcccacatttggtggaacgacaacaattactataacaccgccttgaaaagccgcctcacaatttccaaggatacatccaataaccaagtgctgcttaaaattgccagcgtagattctgctgacactgcaacctattactgtacccgtattgccgacgactatcactttgattattgggggcaaggcacaacagtcatagttagctca

SEQ ID NO: 18 - DNA, 336 bp, synthetic construct
  source 1..336: mol_type = other DNA
gatatagttatgtcacagagtccatcctctctcgccgtgtccaccggggaaaaggtgactatgagttgtaaatcatcacagagtctgttgaactctcgcactcgcaagaattatctcgcatggtaccagcagaaaccaggacaatctccaaagctgctcatttactgggccagtacacgggagtcaggtgttccagaccgcttcactggcagtggaagtggaactgattttacactgaccattagcacagtccagactgaagacttggcagtgtactactgcaaacagagctatcacctctggactttcggcggcggcacaaaacttgagatcaaa

SEQ ID NO: 19 - DNA, 357 bp, synthetic construct
  source 1..357: mol_type = other DNA
gaagtccaagttctcgaatctgggggaggtctcgtacaaccaggcagttcattgaagctttcatgtgctactagcgggtttacattcaataccgcctggatgtactggtatcgtcagtttcctgaaaagaggctcgaatgggtagcccgaatgaaggccaagtccgattcatttgccactgattacagggaaagcgtgaagggacgcttcactatctcccgtgacgactccaaggggtcagtctatctccacatgtataacgtcaaagaagaagacactgcaatttactactgtgcattcgggtatggaacttctggagacgcctggggccagggtgcttctgtcaccgtgtctagc

SEQ ID NO: 20 - DNA, 336 bp, synthetic construct
  source 1..336: mol_type = other DNA
gatgtggttatgactcagaccccacccagtttgagtgttgctatcggccaaagtgttagtatatcatgcaaaagctcacagtccctggtagggcggtcaaaaaacacttatcttaactggtttctgcagtccccaggtcgcagccctaaaaggcttatacatcaagtgtctaaattggatagcggtgttcctgaccgctttagtggcagtggaagcgagcgagactttacacttaaaataagccgtgtagaagctgaagacctgggcgtgtattattgcctccaggggactcatctgcctctgactttcggttctggtacaaaattggaaattaag

SEQ ID NO: 21 - DNA, 357 bp, synthetic construct
  source 1..357: mol_type = other DNA
gaggttcaagtcctggaaagcggtggcgggctcgtgcaacctggttcctcccttaaattgagttgcgccacttctggatttactttcaacacagcctggatgtattggtataggcagttccctgaaaaacgactggagtgggtcgcaagaatgaaagcaaagtcagattccttcgccactgactatcgagaatctgtcaagggtaggttcacaataagtcgcgacgatagcaaggggtcagtctacctgcatatgtataacgtaaaagaggaggataccgccatctattactgcgcttttggttatggtactagcggagacgcttggggccagggtgcttccgtcactgttagttcc

SEQ ID NO: 22 - DNA, 336 bp, synthetic construct
  source 1..336: mol_type = other DNA
gacgtggttatgactcaaactcccccctctctttccgtcgccataggtcagagcgttagcatttcatgtaagtcctcacagtccttggtgggacgaagcaaaaatacttacctcaactggtttctgcaaagcccagggcgttctcccaagagactcatccaccaggtttctaagctggatagtggagttccagatcgttttagtggtagtgggagtgagagagattttacactcaagatttctcgtgtggaggcagaagacctcggagtatattattgcttgcaaggcacacatttgccactgaccttcgggagtgggacaaagctcgaaatcaaa

SEQ ID NO: 23 - protein, 15 aa, synthetic construct
GGGGSGGGGSGGGGS

SEQ ID NO: 24 - protein, 41 aa, synthetic construct
YPYDVPDYAGGGSHGGGGSGGGGSGGGGSGGGGSLGGGSTM

SEQ ID NO: 25 - protein, 51 aa, synthetic construct
HGGGGSGGGGSGGGGSGGGGSLGGSGSAGSAGSGEFYPYDVPDYAGGGSTM

SEQ ID NO: 26 - protein, 41 aa, synthetic construct; identical to SEQ ID NO: 24

SEQ ID NO: 27 - protein, 9 aa, synthetic construct
YPYDVPDYA

SEQ ID NO: 28 - protein, 15 aa, synthetic construct
GGGGSGGGGSGGGGS

SEQ ID NO: 29 - protein, 5 aa, synthetic construct
GGGGS

SEQ ID NO: 30 - protein, 10 aa, synthetic construct
GGGGSGGGGS

SEQ ID NO: 31 - protein, 15 aa, synthetic construct
GGGGSGGGGSGGGGS

SEQ ID NO: 32 - protein, 20 aa, synthetic construct
GGGGSGGGGSGGGGSGGGGS